(19) United States
(12) Patent Application Publication
(10) Pub. No.: US 2009/0299296 A1
(43) Pub. Date: Dec. 3, 2009

(54) CONTAINER FOR RECEIVING MEDICAL LIQUIDS

(76) Inventors: Wolfram Ragg, Arth (CH); Ivan Heutschi, Grosshochstetten (CH); Ulrich Haueter, Grosshochstetten (CH); Thomas Rufer, Ostermundigen (CH); David Bosshard, Bremgarten (CH); Hanspeter Stoller, Bern (CH)

Correspondence Address: Dorsey & Whitney LLP, IP Department, ATTN: Disetronic Licensing AG, 50 South Sixth Street, Suite 1500, Minneapolis, MN, 55402-1498, US

(21) Appl. No.: 12/437,637

(22) Filed: May 8, 2009

Related U.S. Application Data
(63) Continuation of application No. PCT/EP2007/009710, filed on Nov. 9, 2007.

(30) Foreign Application Priority Data
Nov. 10, 2006 (CH) 01774/06

(51) Int. Cl. A61M 5/145; A61B 19/00; A61M 5/315; A61M 5/30

(52) U.S. Cl. 604/208; 604/403; 604/407; 604/246; 604/249

(57) ABSTRACT

A container for receiving liquids with different concentrations of an active substance that is to be administered or dosed, wherein the container includes a hollow body and a displaceable element for expelling the liquid is located for longitudinal movement in the hollow body, and wherein the cross-sectional surface area of the hollow body is inversely proportional to the concentration of the substance, such that, with identical strokes or longitudinal movements of the displaceable element, the discharged amount of active substance remains independent of the concentration of the substance in the container.

### CROSS-REFERENCE TO RELATED APPLICATIONS

This application is a continuation of International Application No. PCT/EP2007/009710, filed on Nov. 9, 2007, which claims priority to Swiss Application No. 01774/061, filed on Nov. 10, 2006, the contents of both of which are incorporated in their entirety herein by reference.

### BACKGROUND

The present invention relates to devices for injecting, infusing, administering, delivering or dispensing a substance, and to methods of making and using such devices. More particularly, it relates to devices for administering or dosing medical, medicinal and/or therapeutic substances or liquids, and to a container for selectively receiving substances or liquids that alternately or selectively contain different concentrations of a substance that is to be dosed or administered.

For treatment of diabetes mellitus, devices of this kind are available, for example, in the form of insulin pumps, injection devices and insulin pens, with which patients can provide themselves with a therapeutic amount of insulin from a storage container associated with the device. The container, for example an ampoule, in which a plunger or stopper is received in a displaceable manner, is fixed in a receiving compartment of the administration device. When the stopper is displaced in an advancing, injection or delivery direction by a drive member of the injection pen or infusion pump, a dose or amount of the substance or liquid is conveyed out of the container through the outlet due to the displacement effect provided by the plunger. To permit individual therapy, such administration appliances generally permit the use of insulin solutions with different insulin concentrations.

The therapeutic or active substance concentrations are generally defined according to international standards. For the insulin concentration, an international unit (IU) corresponds per millimetre to pure human insulin of 26 U/mg (6.00 mmol/U). An insulin unit thus always has the same effect. The insulin concentrations U40 (40 IU/ml=1.5 mg/ml) and U100 (100 IE/ml=3.75 mg/ml) are widely used, while U200 and higher concentrations are increasingly being employed. When using a higher concentration, a smaller amount of liquid has to be delivered to administer the same amount of active substance or the same international units.

Changing from one insulin concentration to another requires adapting or reprogramming the pump or pen. If the reprogramming is not carried out, or is done incorrectly, the wrong amount of active substance enters the human body, which can lead to serious harm or injury to the patient. To ensure patient safety in the medical sector, it is necessary to ensure that no error occurs in the reading of a coding feature that indicates the concentration, something that can never be achieved with absolute certainty, despite considerable outlay in terms of effort and/or construction. Given the serious consequences of a wrongly identified or wrongly programmed insulin concentration, an extremely high safety level must be attained.

In the prior art, injection or infusion devices are known which can identify certain identification means applied to the container, for example bar codes, RFID tags or mechanically acting devices, with the coded insulin concentration and which can automatically adapt the dosing device to the corresponding concentrations. A precondition for this is that the ampoules used are equipped with characteristic identification means or features.

An ampoule or container designed for U200 insulin is disclosed in patent document WO 03/017914. To satisfy the stringent precision requirements regarding the precise amount delivered, a preferred diameter range of the container is disclosed. The preferred dimensions, however, only apply to the U200 insulin concentration.

In patent application WO 05/004954, an adjusting element for adjusting a liquid concentration is described which can be coupled to an ampoule and communicates with an injection device provided for the purpose. The adjustment of the concentration present in the container is to be carried out by the user and can be shown on a display.

### SUMMARY

An object of the present invention is to make available a container from which active substance solutions for different active substance concentrations can be expelled safely and in accordance with a particular or selected therapy, and which does not require adjusting or reprogramming of the active substance concentration on an administration device.

In one embodiment, the present invention comprises a container for receiving liquids with different concentrations of an active substance that is to be dosed, the container comprising a hollow body and a displaceable element for expelling the liquid, the displaceable element located for longitudinal movement in the hollow body, wherein the cross-sectional surface area of the hollow body is inversely proportional to the concentration of the active substance, such that, with identical longitudinal movements of the displaceable element, the dosed amount of active substance remains independent of the concentration.

In one embodiment, the present invention comprises a container for receiving liquids having different concentrations of an active substance that is to be administered or dosed, wherein the container comprises a hollow body and a displaceable element for expelling the liquid is located for longitudinal movement in the hollow body, and wherein the interior cross-sectional surface area of the hollow body is inversely proportional to the concentration of the substance, such that, with identical strokes or longitudinal movements of the displaceable element, the discharged amount of active substance remains independent of the concentration of the substance in the container.

In one embodiment, the present invention comprises a container for receiving medical liquids with different concentrations, which container can be received by an administration device. To permit individual therapy, different insulin concentrations can selectively or alternately be used. In some embodiments, the invention is suitable for use in an infusion or injection device in which a drive member acts on a plunger or stopper that is displaceable in the container. Of course, the cylinder can also be designed to be displaceable relative to the stationary plunger. The substance received in the container can contain a liquid medicament, for example an insulin solution, analgesic agent, nutrient solution or growth hormone, in different concentrations. In some embodiments, the container is arranged releasably in the administration device and can be exchanged when necessary, for example by the physician or patient.

In some embodiments, the container has an axially symmetrical body which has a substantially constant internal cross section and is designed to receive a displaceable element for expelling the liquid. Usually, a displacing body is fitted on or pushed into a front face. The liquid situated in the direction of displacement is expelled by a relative axial movement of the displacing body. Assuming uninterrupted flow conditions between the liquid reservoir and the outlet opening, this results in an expelled liquid volume which is calculated from the product of the cross-sectional surface area and the translational displacement of the displacing body.

If the cross-sectional surface area is chosen to be inversely proportional to the concentration of the substance, then, according to the table below, the delivered amount in insulin units (IU) is constant for identical displacement units (Δx). Each insulin concentration therefore has a so-called characteristic internal diameter. The characteristic internal diameter shown in the table for U100, which represents the most common insulin concentration, corresponds to the ISO standard 13926-1 for pen ampoules.

| | Characteristic | Cross- sectional | Delivered active |
|---|---|---|---|
| Insulin | internal diameter | surface area | substance [IU] at |
| concentration | [mm] | [mm²] | Δx = 1 mm |
| U40 | 15.258 | 182.846 | 7.34 |
| U50 | 13.647 | 146.276 | 7.34 |
| U100 | 9.65 | 73.138 | 7.34 |
| U200 | 6.824 | 36.569 | 7.34 |
| U400 | 4.825 | 18.285 | 7.34 |

Since only the inner cross-sectional surface area or internal diameter influences the amount of active substance delivered, the outer geometry and length dimensions can be chosen freely, within the limits imposed by construction and/or practicality.

In some embodiments, the configuration of the inner cross section has a geometry that is axially symmetrical with respect to the direction of advance, for example a circular, polygonal or elliptic cross section.

In one embodiment, the liquid received by the container selectively includes medical or therapeutic liquids such as insulin for treatment of diabetes mellitus, growth hormones, nutrient solutions or analgesic agents.

In another embodiment, the container is designed for an active substance concentration of a medical liquid of between U10 and U1000.

In one preferred embodiment, the container is formed by an ampoule, carpoule, syringe reservoir or syringe ampoule for containing the substance that is to be dosed. A stopper which is displaceable in the longitudinal direction, and which has a seal or sealing means, for example sealing lips, forming a seal on the cross-sectional surfaces, forms a displacing body with a diameter and cross section. The displaceable stopper is driven by a drive member which ensures that the liquid is delivered at a constant stroke. In other words, each translation of the drive element by one displacement unit effects an expulsion of an identical amount of active substance or international units from the container, independently of the concentration contained in the container.

In one preferred embodiment, the container is a prefilled insulin ampoule in which the insulin concentration contained therein agrees with the characteristic internal diameter and cooperates with a suitable administration device.

In other preferred embodiments, the administration device is an injection pen or an insulin pump permitting self-medication for treatment of diabetes mellitus.

In one embodiment of an administration device, a drive member that can be coupled to the stopper is present. To ensure coupling with different diameter series, the drive member may be adjusted, in some embodiments automatically, to the stopper diameter.

In a preferred embodiment of an administration device, the seal is effected by a stopper which is axially displaceable and can be coupled mechanically to a drive member, wherein the drive member is designed such that it adapts to the diameter of the stopper. With stoppers of large diameter, the stopper may be flexurally rigid to ensure the dimensional stability of the seal and/or to prevent bulging, which may compromise dispensing accuracy.

If the configuration according to the present invention is used in an injection pen, the active substance solution is administered through a cannula or needle mounted or carried on the injection pen. A front end of the cannula protrudes from the injection pen and is able to penetrate the skin. In pump appliances, administration takes place through a catheter line mounted or carried on the outlet. To administer the active substance solution, the drive device initiates a translational movement of the drive member in an advancing or delivery direction. The dispensing procedure is such that, upon actuation of the dispensing mechanism, the plunger is pushed forward by a certain distance in the direction of the outlet of the container, as a result of which the active substance solution is forced into the catheter or into the cannula.

In another preferred embodiment, the internal diameter or the coding on the outer contour of a container to be inserted in an admistration device, e.g. and injection device or insulin pump, is detected by an identification device and the characteristic insulin concentration is calculated. In this way, in addition to the container imprint, the contained concentration can be shown to the user on a reading unit. The determined insulin concentration can be used to optimize the operation and/or performance of the administration device, for example to optimize the response threshold of occlusion detection, which has the purpose of warning the user if the flow in the delivery channel is impeded.

In one preferred embodiment, the drive member is a ram with a releasable connection to the stopper, which is connected with a force fit to the ram before insertion of the active substance container.

### BRIEF DESCRIPTION OF THE DRAWINGS

FIG. 1 shows an embodiment of a container for a low-concentration insulin solution, e.g. U40 or U100, in accordance with the present invention;

FIG. 2 shows an embodiment of a container for insulin solution at a higher concentration, e.g. U200, in accordance with the present invention;

FIG. 3 is a schematic, representational longitudinal section through an embodiment of an administration device, in the form of an infusion device, in accordance with the present invention, and two ampoules which cooperate with the device and receive medical liquids at different concentrations.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc.

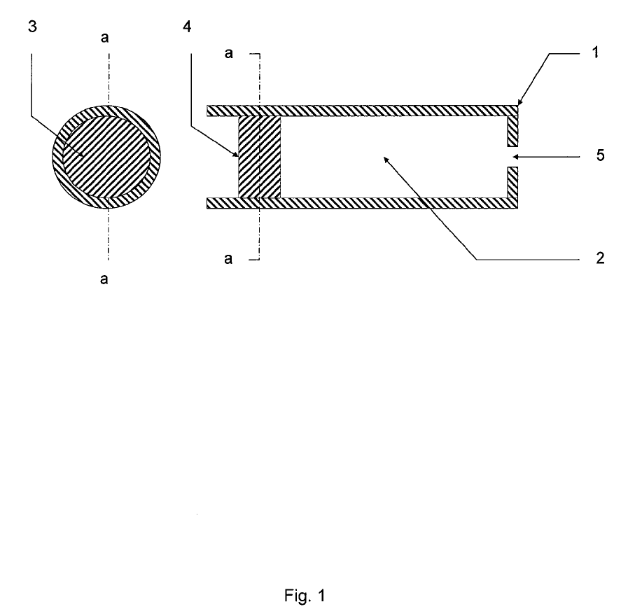

FIG. 1 shows a container (1) in the form of an ampoule which contains a liquid medicament. A radially peripheral seal, which closes off the liquid chamber (2) sealingly on the drive side, ensures a sealed fit of the plunger stopper (4) in the product container. The external diameter of the stopper body is slightly greater than the internal dimensions of the product container, such that the stopper body sits sealingly in the hollow body with elastic deformation and remains displaceable.

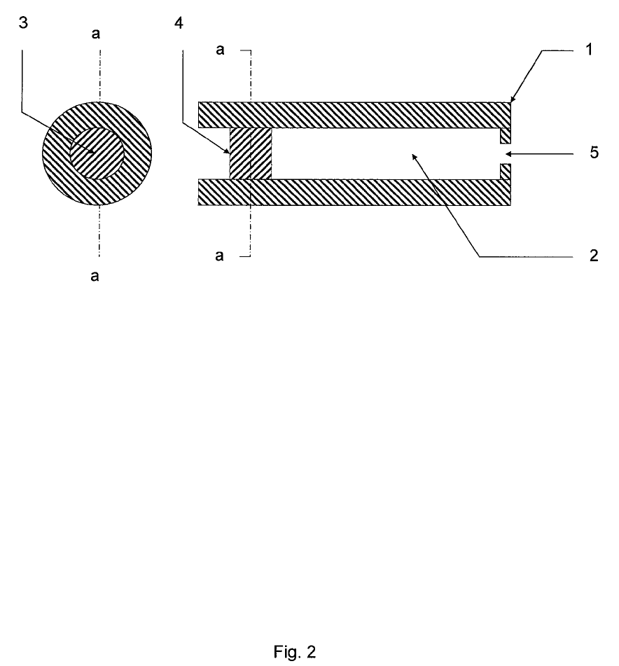

FIG. 2 shows a container (1), in the form of an ampoule, which contains a highly concentrated liquid medicament. If U200 insulin liquid is received in the container, then the cross-sectional surface area is half the cross-sectional surface area of a U100 insulin ampoule. It is generally true that, for containers according to the present invention holding highly concentrated liquids, a smaller diameter is needed than for those holding low-concentration liquids.

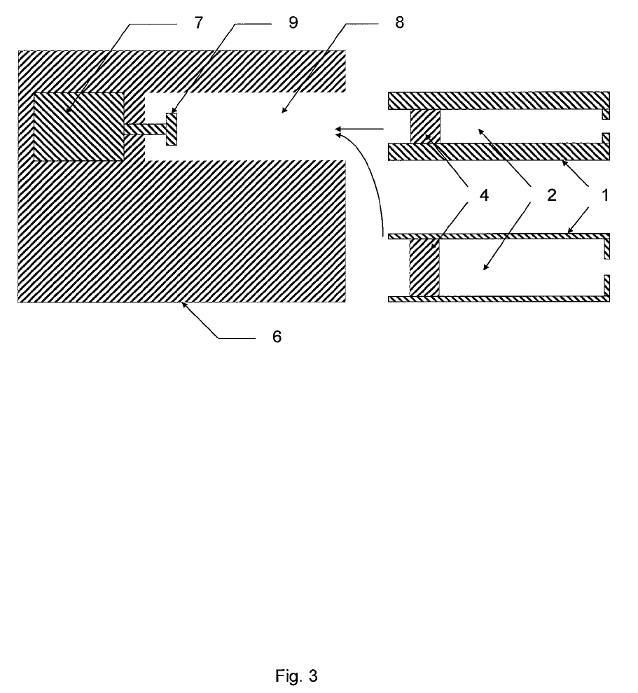

FIG. 3 is a schematic, representational view of an administration device in the form of an infusion device. The infusion device comprises a pump (6) with a drive (7), and a ram (9) which protrudes into the ampoule compartment (8) and allows the stopper (4) to be driven forward along the entire length of the container (1). Different ampoules with various diameter ranges according to the present invention can be received in the ampoule compartment (8). The drive (7) presses on the stopper (4) of the ampoule (1) via the ram (9). The front face of the ram bearing on the stopper is below the smallest internal diameter or characteristic diameter of the alternately or selectively used ampoules or has a diameter compensation element. A diameter compensation element can consist of a cap which can be fitted on the ram, or of a compensation element adjusting to the front face of the stopper.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A container for receiving liquids with different concentrations of an active substance that is to be dosed, the container comprising a hollow body and a displaceable element for expelling the liquid, the displaceable element located for longitudinal movement in the hollow body, wherein the cross-sectional surface area of the hollow body is inversely proportional to the concentration of the active substance, such that, with identical longitudinal movements of the displaceable element, the dosed amount of active substance remains independent of the concentration.

2. A container for receiving liquids with different concentrations of an active substance that is to be dosed, comprising:
a hollow body with a substantially constant internal cross section in the liquid chamber,
an element which is displaceable in the longitudinal direction for expelling the liquid, and
an opening for expelling the liquid, wherein
the internal cross section of the hollow body is inversely proportional to the concentration of the substance, such that, with an identical stroke of the displaceable element, the dosed amount of the active substance to be dosed remains the same.

3. The container according to claim 2, wherein the outer geometry of the container is independent of the geometry of the internal cross section.

4. The container according to claim 2, wherein the internal cross section of the hollow body has one of a constant circular, elliptic or polygonal cross section.

5. The container according to claim 2, wherein the total stroke of the displaceable element is independent of the concentration of the substance to be dosed.

6. The container according to claim 5, wherein the length of the container is at least one of independent of the concentration of the substance to be dosed and constant.

7. The container according to claim 2, wherein the liquid is a medical liquid.

8. The container according to claim 7, wherein the active substance to be dosed is insulin.

9. The container according to claim 8, wherein the insulin has a concentration of between U10 and U1000.

10. The container according to claim 2, wherein the container is an ampoule with a stopper displaceable in a longitudinal direction for expelling the substance to be dosed.

11. The container according to claim 10, wherein the container is suitable for insertion into a device for dosed administration of the medical liquid, and wherein the device comprises one of a portable extracorporeal insulin pump or an insulin pen.

12. The container according to claim 11, wherein the container comprises a primary package for the medical liquid.

13. The container according to claim 2, wherein the container is a prefilled insulin ampoule.

14. A device for for the infusion of insulin, the device comprising an ampoule with a stopper displaceable in a longitudinal direction for expelling the substance to be dosed, wherein the ampoule comprises a hollow body with a substantially constant internal cross section in the liquid chamber, an element which is displaceable in the longitudinal direction for expelling the liquid, and an opening for expelling the liquid, wherein the internal cross section of the hollow body is inversely proportional to the concentration of the substance, such that, with an identical stroke of the displaceable element, the dosed amount of the active substance to be dosed remains the same.

15. The device according to claim 14, wherein the device is a portable insulin pump or an insulin pen.

16. The device according to claim 15, wherein the element comprises a stopper for expelling the medical liquid from the container, and the device further comprises a drive member which can be connected to the stopper for axial displacement of the stopper, said drive member adjusting to the diameter of the stopper.

17. The device according to claim 16, wherein an identification of the internal diameter takes place, and a corresponding insulin concentration is derived from the diameter and is one of associated with the device, stored in or on the device, or indicated on the device.

18. The device according to claim 17, wherein the drive member is a ram.
